(19) United States
(12) Patent Application Publication
(10) Pub. No.: US 2022/0111138 A1
(43) Pub. Date: Apr. 14, 2022

(54) SYSTEMS, APPARATUSES, AND METHODS FOR NEGATIVE-PRESSURE TREATMENT WITH REDUCED TISSUE IN-GROWTH

(71) Applicant: KCI Licensing, Inc., San Antonio, TX (US)

(72) Inventors: Timothy Mark ROBINSON, Shillingstone (GB); Christopher Brian LOCKE, Bournemouth (GB); Diwi L. ALLEN, San Antonio, TX (US)

(21) Appl. No.: 17/275,507

(22) PCT Filed: Sep. 12, 2019
(86) PCT No.: PCT/US2019/050880

§ 371 Date: Mar. 11, 2021

Related U.S. Application Data
(60) Provisional application No. 62/730,332, filed on Sep. 12, 2018.

(51) Int. Cl. A61M 1/00

(52) U.S. Cl. CPC A61M 1/915

(57) ABSTRACT

A dressing for treating a tissue site with negative pressure includes a manifold having a first surface and a second surface opposite the first surface. The dressing also includes a first layer adjacent to the first surface and a second layer adjacent to the second surface. The first layer and the second layer each include a polymer film. The dressing also includes a plurality of fluid restrictions in the polymer film adjacent to at least the first surface, and a plurality of bonds between the first layer and the second layer. The plurality of bonds defines separable sections of the manifold. Each of the separable sections of the manifold has at least one rounded edge.

### RELATED APPLICATION

The present application claims priority to U.S. Provisional Patent Application No. 62/730,332, entitled “Systems, Apparatuses, And Methods For Negative-Pressure Treatment With Reduced Tissue In-Growth,” filed Sep. 12, 2018, which is incorporated herein by reference for all purposes.

### TECHNICAL FIELD

The invention set forth in the appended claims relates generally to tissue treatment systems and more particularly, but without limitation, to systems, dressings, and fillers for negative-pressure tissue treatment, and methods of using systems, dressings, and fillers for negative-pressure tissue treatment.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound can be washed out with a stream of liquid solution, or a cavity can be washed out using a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and instillation therapy are widely known, improvements to therapy systems, components, and processes may benefit healthcare providers and patients.

### BRIEF SUMMARY

New and useful systems, apparatuses, and methods for treating tissue in a negative-pressure therapy environment are set forth in the appended claims. Illustrative embodiments are also provided to enable a person skilled in the art to make and use the claimed subject matter.

For example, in some embodiments, a dressing or filler for treating a tissue site with negative-pressure may include a thin sheet of reticulated foam enclosed within at least two layers of perforated or fenestrated film. Suitable films may include, for example, polythene, polyurethane, or ethyl methyl acrylate. Some embodiments of the film may have linear perforations or fenestrations formed over the surface. The foam may be formed in sections in some embodiments. For example, the foam sections may be formed by joining the film layer around the sections. Sections may be formed having rounded edges in some examples, and the composite of foam and film may resemble a quilted structure in some configurations. Sections may be folded, cut, or otherwise separated to shape and size the dressing or filler for optimal placement, and exposure of the foam may be avoided or minimized by folding or separating the sections along joined film layers between sections.

More generally, in some embodiments, a dressing for treating a tissue site with negative pressure may include a manifold comprising a first surface and a second surface opposite the first surface, a first layer adjacent to the first surface and a second layer adjacent to the second surface, the first layer and the second layer each comprising a polymer film, a plurality of fluid restrictions in the polymer film adjacent to at least the first surface, and a plurality of bonds between the first layer and the second layer. The plurality of bonds may define separable sections of the manifold. Each of the separable sections of the manifold have at least one rounded edge.

In some embodiments, the plurality of bonds form seams between the separable sections of the manifold. The seams between the sections generally have a width of at least 2 millimeters. The manifold may also include perforations, which may be aligned with the bonds in some examples. The manifold may additionally include sacrificial joints or bonds between the separable sections in some embodiments.

In some embodiments, the plurality of bonds may define separable sections as circular shapes, spiral shapes, or oval shapes. In other embodiments, the plurality of bonds may define separable sections having only acute angles, only obtuse angles, or no right angles.

Some embodiments of a dressing may include a dressing for treating a tissue site with negative pressure including a manifold comprising a first surface and a second surface opposite the first surface, a first layer adjacent to the first surface and a second layer adjacent to the second surface, the first layer and the second layer each comprising a polymer film, a plurality of fluid restrictions in the polymer film adjacent to at least the first surface, and a means for bonding the first layer to the second layer to form separable sections of the manifold, the separable section having at least one rounded edge. In some embodiments, the means for bonding the first layer to the second layer may include at least one of a weld or a bond.

In some embodiments, a dressing for treating a tissue site with negative pressure may include a manifold comprising a first surface and a second surface opposite the first surface, a first layer adjacent to the first surface and a second layer adjacent to the second surface, the first layer and the second layer each comprising a polymer film, a plurality of fluid restrictions in the polymer film adjacent to at least the first surface, and a means for bonding the first layer to the second layer to form separable sections of the manifold. The separable section may each have no right angles therein.

In other embodiments, a dressing for treating a tissue site with negative pressure may comprise a first manifold layer and a second manifold layer, at least one intermediate layer between the first manifold layer and the second manifold layer, a first outer layer adjacent to the first manifold layer opposite the intermediate layer, a second outer layer adjacent to the second manifold layer opposite the intermediate layer, a plurality of fluid restrictions in the first outer layer and the second outer layer, and a plurality of bonds between the first outer layer and the intermediate layer and between the second outer layer and the intermediate layer. At least the first outer layer and the second outer layer may each comprise a polymer film. The plurality of bonds may define separable sections of the first manifold layer and the second manifold layer. The separable sections may each have at least one rounded edge and no right angles therein.

Other embodiments may relate to an a dressing for treating a tissue site with negative pressure, the dressing comprising a manifold comprising a first surface and a second surface opposite the first surface, a first layer adjacent to the first surface and a second layer adjacent to the second surface, the first layer and the second layer each comprising a polymer film, a plurality of fluid restrictions in the polymer film adjacent to at least the first surface, and a plurality of bonds between the first layer and the second layer. The plurality of bonds may define separable sections of the manifold. Each of the separable sections of the manifold may be in the form of polygons having more than five edges. In some embodiments, the plurality of bonds may define separable sections having hexagonal shapes.

In some embodiments, an apparatus for providing negative-pressure treatment to a tissue site may comprise a manifold comprising a first surface and a second surface opposite the first surface, a first layer adjacent to the first surface and a second layer adjacent to the second surface, the first layer and the second layer each comprising a polymer film, a plurality of fluid restrictions in the polymer film adjacent to at least the first surface, a plurality of bonds between the first layer and the second layer, the plurality of bonds defining separable sections of the manifold, the separable sections each having at least one rounded edge, and a negative-pressure source fluidly coupled to the manifold.

Other example embodiments may relate to a method for treating a tissue site. The method may comprise excising separable sections of a dressing based upon at least one of a size and shape of the tissue site, each of the separable sections having at least one rounded edge, applying the dressing to fill or cover the tissue site, sealing the dressing to epidermis adjacent to the tissue site, fluidly coupling the dressing to a negative-pressure source, and applying negative pressure from the negative-pressure source to the dressing.

In some embodiments, the separable shapes are in the form of rounded shapes that, when torn, may not leave acute corners so as to reduce irritation or discomfort at a wound. In other embodiments, the separable shapes may be brightly colored so as to be more noticeable and easier to locate in deep wounds.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide tissue treatment in accordance with this specification.

FIG. 2 is an exploded view of a dressing that may be associated with an example embodiment of the therapy system of FIG. 1.

FIG. 3 is a top view of a tissue interface of the dressing of FIG. 2.

FIG. 4 is a cross-sectional view of the tissue interface of FIG. 3.

FIG. 5 is a top view of another example of a tissue interface that may be associated with some embodiments of the dressing of FIG. 2.

FIG. 6 is a top view of another example of a tissue interface that may be associated with some embodiments of the dressing of FIG. 2.

FIG. 7 is a top view of another example of a tissue interface that may be associated with some embodiments of the dressing of FIG. 2.

FIG. 8 is a top view of another example of a tissue interface that may be associated with some embodiments of the dressing of FIG. 2.

FIG. 9 is a top view of another example of a tissue interface that may be associated with some embodiments of the dressing of FIG. 2.

FIG. 10 is a top view of another example of a tissue interface that may be associated with some embodiments of the dressing of FIG. 2.

FIG. 11 is a top view of another example of a tissue interface that may be associated with some embodiments of the dressing of FIG. 2.

FIG. 12 is a top view of another example of a tissue interface that may be associated with some embodiments of the dressing of FIG. 2.

FIG. 13 is a top view of another example of a tissue interface that may be associated with some embodiments of the dressing of FIG. 2.

FIG. 14 is a top view of another example of a tissue interface that may be associated with some embodiments of the dressing of FIG. 2.

FIG. 15 is a top view of another example of a tissue interface that may be associated with some embodiments of the dressing of FIG. 2.

FIG. 16 is a top view of another example of a tissue interface that may be associated with some embodiments of the dressing of FIG. 2.

FIG. 17 an exploded view of another example tissue interface that may be associated with some embodiments of the therapy system of FIG. 1.

FIG. 18 is a schematic view of an example configuration of a layer that may be associated with some embodiments of the tissue interface of FIG. 17.

FIG. 19 is a schematic view of the example layer of FIG. 18 overlaid on the tissue interface of FIG. 3.

FIG. 20 is a schematic view of another example of another dressing layer, illustrating additional details that may be associated with some embodiments.

FIG. 21 is a schematic view of another example of another dressing layer, illustrating additional details that may be associated with some embodiments.

FIG. 22 is a schematic view of another example of another dressing layer, illustrating additional details that may be associated with some embodiments.

FIG. 23 is a section view of another example tissue interface that may be associated with some embodiments of the therapy system of FIG. 1.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

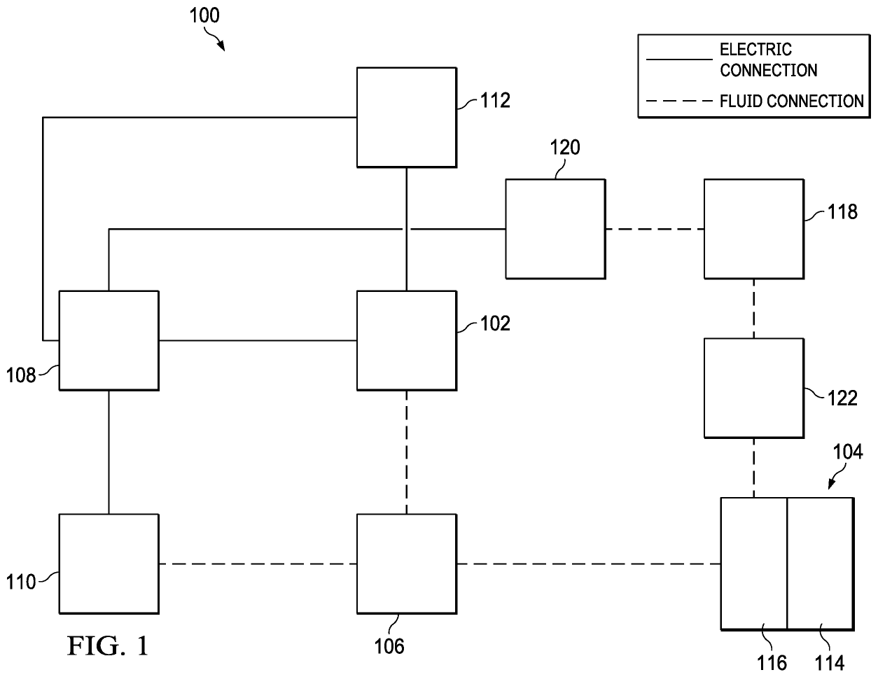

FIG. 1 is a simplified functional block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including but not limited to, a surface wound, bone tissue, adipose tissue, muscle tissue, neural tissue, dermal tissue, vascular tissue, connective tissue, cartilage, tendons, or ligaments. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted. A surface wound, as used herein, is a wound on the surface of a body that is exposed to the outer surface of the body, such an injury or damage to the epidermis, dermis, and/or subcutaneous layers. Surface wounds may include ulcers or closed incisions, for example. A surface wound, as used herein, does not include wounds within an intra-abdominal cavity. A wound may include chronic, acute, traumatic, subacute, and dehisced wounds, partial-thickness burns, ulcers (such as diabetic, pressure, or venous insufficiency ulcers), flaps, and grafts, for example.

The therapy system 100 may include a source or supply of negative pressure, such as a negative-pressure source 102, a dressing 104, a fluid container, such as a container 106, and a regulator or controller, such as a controller 108, for example. Additionally, the therapy system 100 may include sensors to measure operating parameters and provide feedback signals to the controller 108 indicative of the operating parameters. As illustrated in FIG. 1, for example, the therapy system 100 may include a pressure sensor 110, an electric sensor 112, or both, coupled to the controller 108. As illustrated in the example of FIG. 1, the dressing 104 may comprise or consist essentially of a tissue interface 114, a cover 116, or both in some embodiments.

The therapy system 100 may also include a source of instillation solution. For example, a solution source 118 may be fluidly coupled to the dressing 104, as illustrated in the example embodiment of FIG. 1. The solution source 118 may be fluidly coupled to a positive-pressure source such as the positive-pressure source 120, a negative-pressure source such as the negative-pressure source 102, or both in some embodiments. A regulator, such as an instillation regulator 122, may also be fluidly coupled to the solution source 118 and the dressing 104 to ensure proper dosage of instillation solution (e.g. saline) to a tissue site. For example, the instillation regulator 122 may comprise a piston that can be pneumatically actuated by the negative-pressure source 102 to draw instillation solution from the solution source during a negative-pressure interval and to instill the solution to a dressing during a venting interval. Additionally or alternatively, the controller 108 may be coupled to the negative-pressure source 102, the positive-pressure source 120, or both, to control dosage of instillation solution to a tissue site. In some embodiments, the instillation regulator 122 may also be fluidly coupled to the negative-pressure source 102 through the dressing 104, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 102 may be combined with the solution source 118, the controller 108, and other components into a therapy unit.

In general, components of the therapy system 100 may be coupled directly or indirectly. For example, the negative-pressure source 102 may be directly coupled to the container 106, and may be indirectly coupled to the dressing 104 through the container 106. Coupling may include fluid, mechanical, thermal, electrical, or chemical coupling (such as a chemical bond), or some combination of coupling in some contexts. For example, the negative-pressure source 102 may be electrically coupled to the controller 108, and may be fluidly coupled to one or more distribution components to provide a fluid path to a tissue site. In some embodiments, components may also be coupled by virtue of physical proximity, being integral to a single structure, or being formed from the same piece of material. For example, the tissue interface 114 and the cover 116 may be discrete layers disposed adjacent to each other, and may be joined together in some embodiments.

A distribution component is preferably detachable, and may be disposable, reusable, or recyclable. The dressing 104 and the container 106 are illustrative of distribution components. A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 104.

A negative-pressure supply, such as the negative-pressure source 102, may be a reservoir of air at a negative pressure, or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 106 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 108, may be a microprocessor or computer programmed to operate one or more components of the therapy system 100, such as the negative-pressure source 102. In some embodiments, for example, the controller 108 may be a microcontroller, which generally comprises an integrated circuit containing a processor core and a memory programmed to directly or indirectly control one or more operating parameters of the therapy system 100. Operating parameters may include the power applied to the negative-pressure source 102, the pressure generated by the negative-pressure source 102, or the pressure distributed to the tissue interface 114, for example. The controller 108 is also preferably configured to receive one or more input signals, such as a feedback signal, and programmed to modify one or more operating parameters based on the input signals.

Sensors, such as the pressure sensor 110 or the electric sensor 112, are generally known in the art as any apparatus operable to detect or measure a physical phenomenon or property, and generally provide a signal indicative of the phenomenon or property that is detected or measured. For example, the pressure sensor 110 and the electric sensor 112 may be configured to measure one or more operating parameters of the therapy system 100. In some embodiments, the pressure sensor 110 may be a transducer configured to measure pressure in a pneumatic pathway and convert the measurement to a signal indicative of the pressure measured. In some embodiments, for example, the pressure sensor 110 may be a piezoresistive strain gauge. The electric sensor 112 may optionally measure operating parameters of the negative-pressure source 102, such as the voltage or current, in some embodiments. Preferably, the signals from the pressure sensor 110 and the electric sensor 112 are suitable as an input signal to the controller 108, but some signal conditioning may be appropriate in some embodiments. For example, the signal may need to be filtered or amplified before it can be processed by the controller 108. Typically, the signal is an electrical signal, but may be represented in other forms, such as an optical signal.

The tissue interface 114 can be generally adapted to partially or fully contact a tissue site. The tissue interface 114 may take many forms, and may have many sizes, shapes, or thicknesses depending on a variety of factors, such as the type of treatment being implemented or the nature and size of a tissue site. For example, the size and shape of the tissue interface 114 may be adapted to the contours of deep and irregular shaped tissue sites.

In some embodiments, the cover 116 may provide a bacterial barrier and protection from physical trauma. The cover 116 may also be constructed from a material that can reduce evaporative losses and provide a fluid seal between two components or two environments, such as between a therapeutic environment and a local external environment. The cover 116 may comprise or consist essentially of, for example, an elastomeric film or membrane that can provide a seal adequate to maintain a negative pressure at a tissue site for a given negative-pressure source. The cover 116 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 116 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 116 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minn.; polyurethane (PU) drape, commercially available from Avery Dennison Corporation, Pasadena, Calif.; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 116 may comprise INSPIRE 2301 having an MVTR (upright cup technique) of 2600 g/m²/24 hours and a thickness of about 30 microns.

An attachment device may be used to attach the cover 116 to an attachment surface, such as undamaged epidermis, a gasket, or another cover. The attachment device may take many forms. For example, an attachment device may be a medically-acceptable, pressure-sensitive adhesive configured to bond the cover 116 to epidermis around a tissue site. In some embodiments, for example, some or all of the cover 116 may be coated with an adhesive, such as an acrylic adhesive, which may have a coating weight between 25-65 grams per square meter (g·s·m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 118 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications (such as by substituting a positive-pressure source for a negative-pressure source) and this descriptive convention should not be construed as a limiting convention.

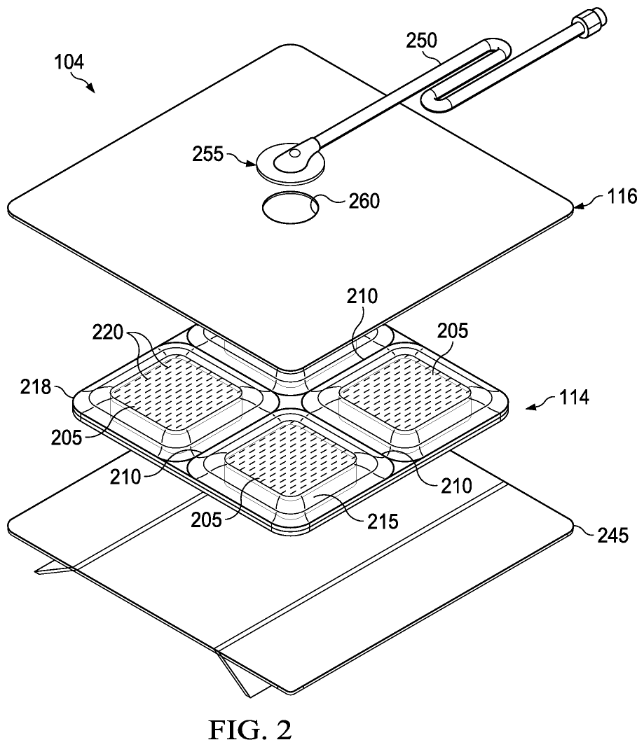

FIG. 2 is an assembly view of an example of the dressing 104 of FIG. 1, illustrating additional details that may be associated with some embodiments in which the tissue interface 114 comprises separable sections. In the example of FIG. 2, the tissue interface 114 comprises one or more interface sections 205, which may be bounded by seams 210. Each of the interface sections 205 may include a manifold section 215. In some examples, seams 210 may be formed between or may define the manifold sections 215. Each of the interface sections 205 may have one or more seams 210, edges 218, or both that are rounded. In some examples, all of the edges 218 may be rounded or have rounded corners.

The manifold sections 215 may comprise or consist of foam in some embodiments. For example, the foam may be open-cell foam, such as reticulated foam. The foam may also be relatively thin and hydrophobic to reduce the fluid hold capacity of the dressing, which can encourage exudate and other fluid to pass quickly to external storage. The foam layer may also be thin to reduce the dressing profile and increase flexibility, which can enable it to conform to wound beds and other tissue sites under negative pressure. In some embodiments, the manifold sections 215 may be formed of 3-dimensional textiles, non-woven wicking material, vacuum-formed texture surfaces, and composites thereof. A hydrophobic manifold having a thickness of less than 7 millimeters and a free volume of at least 90% may be suitable for many therapeutic applications. In some embodiments, the manifold sections 215 may be formed of colored material, such that the manifold sections 215 are easily located when in or adjacent wounds. Each of the manifold sections 215 may be a same color or a different color.

As illustrated in the example of FIG. 2, the tissue interface 114 may have one or more fluid restrictions 220, which can be distributed uniformly or randomly across the tissue interface 114. The fluid restrictions 220 may be bi-directional and pressure-responsive. For example, each of the fluid restrictions 220 generally may comprise or consist essentially of an elastic passage that is normally unstrained to substantially reduce liquid flow, and can expand or open in response to a pressure gradient. The fluid restrictions 220 may be coextensive with the manifold sections 215.

For example, some embodiments of the fluid restrictions 220 may comprise or consist essentially of one or more slits, slots or combinations of slits and slots. In some examples, the fluid restrictions 220 may comprise or consist of linear slots having a length less than 4 millimeters and a width less than 1 millimeter. The length may be at least 2 millimeters, and the width may be at least 0.4 millimeters in some embodiments. A length of about 3 millimeters and a width of about 0.8 millimeters may be particularly suitable for many applications, and a tolerance of about 0.1 millimeter may also be acceptable. Such dimensions and tolerances may be achieved with a laser cutter, for example. In some embodiments, the fluid restrictions 220 may be formed by ultrasonics or other heat means. Slots of such configurations may function as imperfect valves that substantially reduce liquid flow in a normally closed or resting state. For example, such slots may form a flow restriction without being completely closed or sealed. The slots can expand or open wider in response to a pressure gradient to allow increased liquid flow.

As illustrated in the example of FIG. 2, in some embodiments, the dressing 104 may include a release liner 245 to protect an optional adhesive on a portion of the cover 116 prior to use. The release liner 245 may also provide stiffness to assist with, for example, deployment of the dressing 104. The release liner 245 may be, for example, a casting paper, a film, or polyethylene. Further, in some embodiments, the release liner 245 may be a polyester material such as polyethylene terephthalate (PET), or similar polar semi-crystalline polymer. The use of a polar semi-crystalline polymer for the release liner 245 may substantially preclude wrinkling or other deformation of the dressing 104. For example, the polar semi-crystalline polymer may be highly orientated and resistant to softening, swelling, or other deformation that may occur when brought into contact with components of the dressing 104, or when subjected to temperature or environmental variations, or sterilization. Further, a release agent may be disposed on a side of the release liner 245 that is configured to contact the tissue interface 114. For example, the release agent may be a silicone coating and may have a release factor suitable to facilitate removal of the release liner 245 by hand and without damaging or deforming the dressing 104. In some embodiments, the release agent may be a fluorocarbon or a fluorosilicone, for example. In other embodiments, the release liner 245 may be uncoated or otherwise used without a release agent.

FIG. 2 also illustrates one example of a fluid conductor 250 and a dressing interface 255. As shown in the example of FIG. 2, the fluid conductor 250 may be a flexible tube, which can be fluidly coupled on one end to the dressing interface 255. The dressing interface 255 may be an elbow connector, as shown in the example of FIG. 2, which can be placed over an aperture 260 in the cover 116 to provide a fluid path between the fluid conductor 250 and the tissue interface 114.

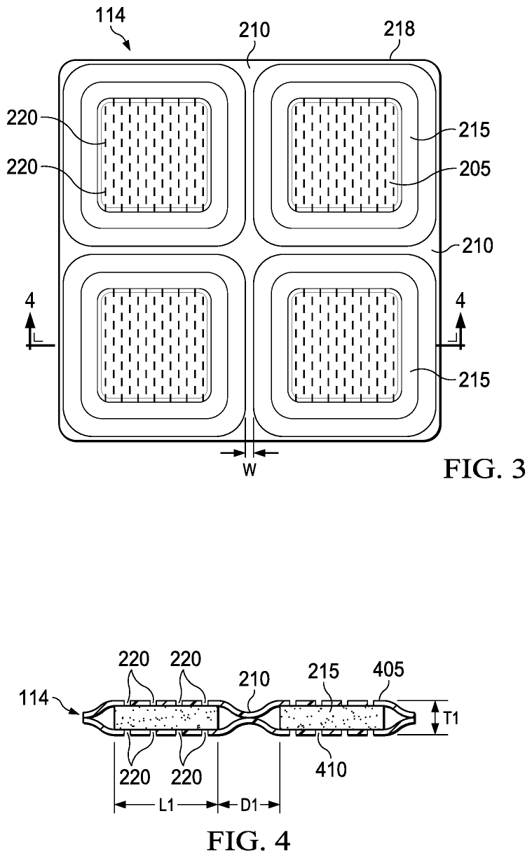

FIG. 3 is a top view of the tissue interface 114 of FIG. 2, illustrating additional details that may be associated with some examples. The manifold sections 215 in each of the interface sections 205 may have a same shape or a different shape. As shown in the example of FIG. 3, the interface sections 205 and the manifold sections 215 may have similar shapes. In some embodiments, the interface sections 205, the manifold sections 215, or both may be elliptical or circular. For example, the interface sections 205 and the manifold sections 215 may be generally square with rounded corners, such as the squircles shown in FIG. 3. More generally, the interface sections 205, the manifold sections 215, or both may have a variety of shapes with no acute vertices. In some embodiments, the profile may be a regular polygon having at least five sides. The manifold sections 215 may have dimensions ranging from about 10 mm to about 40 mm. For example, the manifold sections 215 may be generally shaped as squircles, each having dimensions of about 20 mm by 20 mm.

Each of the seams 210 may have a width W ranging from about 2 mm to about 5 mm, and may be wide enough to allow for the interface sections 205 to be separated along the seams 210 without exposing any portion of the manifold sections 215.

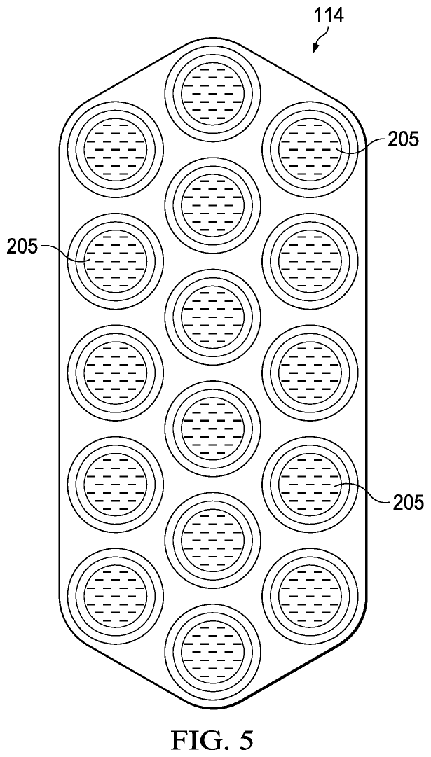

FIG. 4 is a section view of the tissue interface 114 of FIG. 3 taken along line 4-4, illustrating additional details that may be associated with some embodiments. In the example of FIG. 4, the tissue interface 114 comprises a first layer 405, a second layer 410, and the manifold sections 215 disposed between the first layer 405 and the second layer 410. In some embodiments, the first layer 405 and the second layer 410 may be disposed adjacent to the manifold sections 215 as shown in the example of FIG. 4. Also as shown in the example of FIG. 4, the seams 210 may be formed by one or more bonds between the first layer 405 and the second layer 410. The bonds may be continuous or discrete. The tear strength of each of the bonds may vary, but a tear strength in a range of about 1 newton to about 2.5 newtons may be advantageous for some examples. Collectively, the bonds may provide a tear strength of about 10 newtons to about 25 newtons in some examples.

The first layer 405 and the second layer 410 may comprise or consist essentially of a means for controlling or managing fluid flow. In some embodiments, the first layer 405 and the second layer 410 may comprise or consist essentially of an elastomeric material that is impermeable to liquid. For example, the first layer 405 and the second layer 410 may comprise or consist essentially of a polymer film. The first layer 405 and the second layer 410 may also have a smooth or matte surface texture in some embodiments. A glossy or shiny finish better or equal to a grade B3 according to the SPI (Society of the Plastics Industry) standards may be particularly advantageous for some applications. In some embodiments, variations in surface height may be limited to acceptable tolerances. For example, the surface of the second layer may have a substantially flat surface, with height variations limited to 0.2 millimeters over a centimeter.

In some embodiments, the first layer 405 and the second layer 410 may comprise or consist essentially of a hydrophobic material. The hydrophobicity may vary, but may have a contact angle with water of at least ninety degrees in some embodiments. In some embodiments the hydrophobic material may have a contact angle with water of no more than 150 degrees. For example, in some embodiments, the contact angle may be in a range of at least 90 degrees to about 120 degrees, or in a range of at least 120 degrees to 150 degrees. Water contact angles can be measured using any standard apparatus. Although manual goniometers can be used to visually approximate contact angles, contact angle measuring instruments can often include an integrated system involving a level stage, liquid dropper such as a syringe, camera, and software designed to calculate contact angles more accurately and precisely, among other things. Non-limiting examples of such integrated systems may include the FTÅ125, FTÅ200, FTÅ2000, and FTÅ4000 systems, all commercially available from First Ten Angstroms, Inc., of Portsmouth, Va., and the DTA25, DTA30, and DTA100 systems, all commercially available from Kruss GmbH of Hamburg, Germany. Unless otherwise specified, water contact angles herein are measured using deionized and distilled water on a level sample surface for a sessile drop added from a height of no more than 5 cm in air at 20-25° C. and 20-50% relative humidity. Contact angles reported herein represent averages of 5-9 measured values, discarding both the highest and lowest measured values. The hydrophobicity of the first layer 405, the second layer 410, or both may be further enhanced with a hydrophobic coating of other materials, such as silicones and fluorocarbons, either as coated from a liquid, or plasma coated.

The first layer 405 and the second layer 410 may also be suitable for bonding to other layers, including each other. For example, the first layer 405, the second layer 410, or both may be adapted for welding to polyurethane foams using heat, radio frequency (RF) welding, or other methods to generate heat such as laser or ultrasonic welding. RF welding may be particularly suitable for more polar materials, such as polyurethane, polyamides, polyesters and acrylates. Sacrificial polar interfaces may be used to facilitate RF welding of less polar film materials, such as polyethylene. The first layer 405 and the second layer 410 may include hot melt films.

The area density of the first layer 405 and the second layer 410 may vary according to a prescribed therapy or application. In some embodiments, an area density of less than 40 grams per square meter may be suitable, and an area density of about 20-30 grams per square meter may be particularly advantageous for some applications.

In some embodiments, for example, the first layer 405, the second layer 410, or both may comprise or consist essentially of a hydrophobic polymer, such as a polyethylene film. The simple and inert structure of polyethylene can provide a surface that interacts little, if any, with biological tissues and fluids, providing a surface that may encourage the free flow of liquids and low adherence, which can be particularly advantageous for many applications. Other suitable polymeric films include polyurethanes, acrylics, polyolefin (such as cyclic olefin copolymers), polyacetates, polyamides, polyesters, copolyesters, PEBAX block copolymers, thermoplastic elastomers, thermoplastic vulcanizates, polyethers, polyvinyl alcohols, polypropylene, polymethylpentene, polycarbonate, styreneics, silicones, fluoropolymers, and acetates. A thickness between 20 microns and 100 microns may be suitable for many applications. Films may be clear, colored, or printed. More polar films suitable for laminating to a polyethylene film include polyamide, co-polyesters, ionomers, and acrylics. To aid in the bond between a polyethylene and polar film, tie layers may be used, such as ethylene vinyl acetate, or modified polyurethanes. An ethyl methyl acrylate (EMA) film may also have suitable hydrophobic and welding properties for some configurations.

In some embodiments, the fluid restrictions 220 may comprise or consist essentially of perforations in at least one of the first layer 405 and the second layer 410. Perforations may be formed by removing material from the first layer 405, the second layer 410, or both. For example, perforations may be formed by cutting through the material, which may also deform the edges of the perforations in some embodiments. In the absence of a pressure gradient across the perforations, the passages may be sufficiently small to form a seal or fluid restriction, which can substantially reduce or prevent liquid flow. Additionally or alternatively, one or more of the fluid restrictions 220 may be an elastomeric valve that is normally closed when unstrained to substantially prevent liquid flow, and can open in response to a pressure gradient. A fenestration in the material may be a suitable valve for some applications. Fenestrations may also be formed by removing material, but the amount of material removed and the resulting dimensions of the fenestrations may be an order of magnitude less than perforations, and may not deform the edges. In some embodiments, the fluid restrictions 220 extend through both the first layer 405 and the second layer 410, and the fluid restrictions 220 are coextensive with at least one of the first layer 405 and the second layer 410.

Each of the manifold sections 215 has a length L1, which can be in a range from about 10 mm to about 30 mm (e.g., about 15 mm to about 25 mm or about 18 mm to about 22 mm). For example, each of the manifold sections 215 may have a length of about 20 mm. In some embodiments, the manifold sections 215 may be spaced apart by a distance D1 of about 5 mm to about 15 mm. For example, a distance D1 of about 10 mm may be particularly advantageous for some embodiments.

In some embodiments, each of the manifold sections 215 in the tissue interface 114 may be the same size. In other embodiments, one or more of the manifold sections 215 in the tissue interface 114 may have a different size.

In some embodiments, the tissue interface 114 has a thickness T1 ranging from about 5 mm to about 20 mm (e.g., about 8 mm to about 18 mm, or about 10 mm to about 15 mm). For example, the tissue interface 114 may have a thickness T1 of about 8 mm. The thickness T1 of the tissue interface 114 may vary depending upon a thickness of the manifold sections 215 used to form the tissue interface 114. For example, each of the manifold sections 215 may have a thickness ranging from about 5 mm to about 15 mm (e.g., about 8 mm to about 12 mm).

In some embodiments, the first layer 405 and the second layer 410 may be formed of a transparent polymer to aid in cutting the interface sections 205 apart along the seams 210.

In some embodiments, the tissue interface 114 can be formed by spacing the manifold sections 215 apart, placing the first layer 405 of polymer film over the manifold sections 215, placing the second layer 410 under the manifold sections 215, and bonding the first layer 405 to the second layer 410, forming the seams 210 between the manifold sections 215. Suitable means for bonding the first layer 405 to the second layer 410 may include, for example, an adhesive such as an acrylic, and welding, such as heat, radio frequency (RF), laser, or ultrasonic welding. In some embodiments, sacrificial materials may be disposed between the first layer 405 and the second layer 410 to facilitate welding. Suitable sacrificial materials may include, for example, hot melt films supplied by Bayer (such as H2, HU2, and H5 films), Cornelius (Collano film), or Prochimir (such as TC203 or TC206 film).

In some embodiments, the manifold sections 215 may be formed from an integral manifold material, such as foam. In some embodiments, for example, bonds between the first layer 405 and the second layer 410 may extend through a layer of manifold material to define the manifold sections 215. For example, some embodiments of a manifold layer may have a thickness ranging from about 5 mm to about 8 mm, and at least one of the first layer 405 and the second layer 410 may melt through the manifold layer during welding to form the seams 210.

Additionally or alternatively, a unitary manifold material can be perforated and cut to define the manifold sections 215 in a variety of suitable shapes and patterns. In some embodiments, the seams 210 may align with perforations between the manifold sections 215. In some examples, sacrificial joints may be left between the manifold sections 215 to maintain the manifold sections 215 together as a single unit. Maintaining the manifold sections 215 as a single unit can allow for easier assembly of the tissue interface 114. In some embodiments, either or both of the first layer 405 and the second layer 410 may also be bonded to the manifold sections 215 for additional stability.

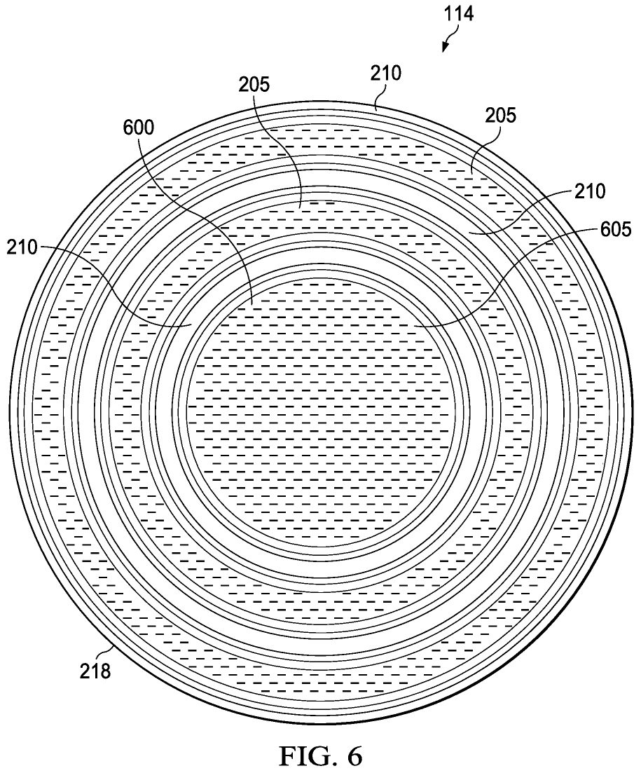

FIG. 5 is a top view of another example of the tissue interface 114, illustrating additional details that may be associated with some embodiments. In the example of FIG. 5, each of the interface sections 205 have generally circular shapes. The interface sections 205 may each have a same diameter or a different diameter. In the example of FIG. 5, the interface sections 205 each have a circular profile with substantially equal diameters. As shown in FIG. 5, the interface sections 205 may be tiled uniformly and may be arranged to maximize the packing density of the interface sections 205. A hexagonal packing of the interface sections 205 may be suitable for some examples. The interface sections 205 may have a diameter of about 10 mm to about 30 mm (e.g., about 15 mm to about 25 mm or about 18 mm to about 22 mm). For example, each of the interface sections 205 may have a diameter of about 20 mm.

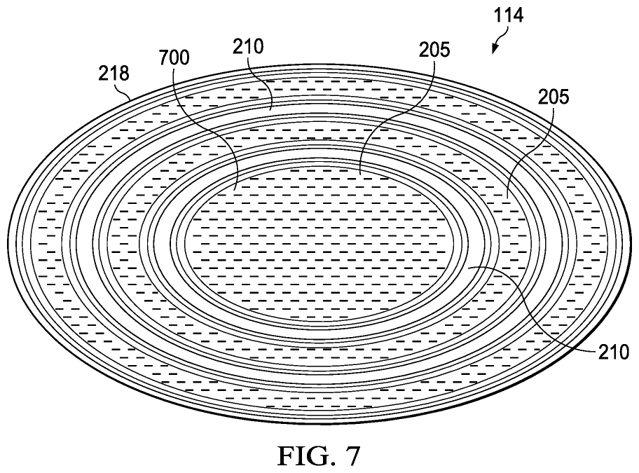

FIG. 6 is a top view of another example of the tissue interface 114, illustrating additional details that may be associated with some embodiments. The interface sections 205 may have nested inner and outer edges, which may be concentric or non-concentric. In the example of FIG. 6, the interface sections 205 are bounded by seams 210 that are circular and concentric. In other examples, the seams 210 may define circles having collinear centers. The seams 210 may be spaced equally or variably from the center of the tissue interface 114. For example, the seams 210 may form outer rings that are thicker than inner rings in some embodiments.

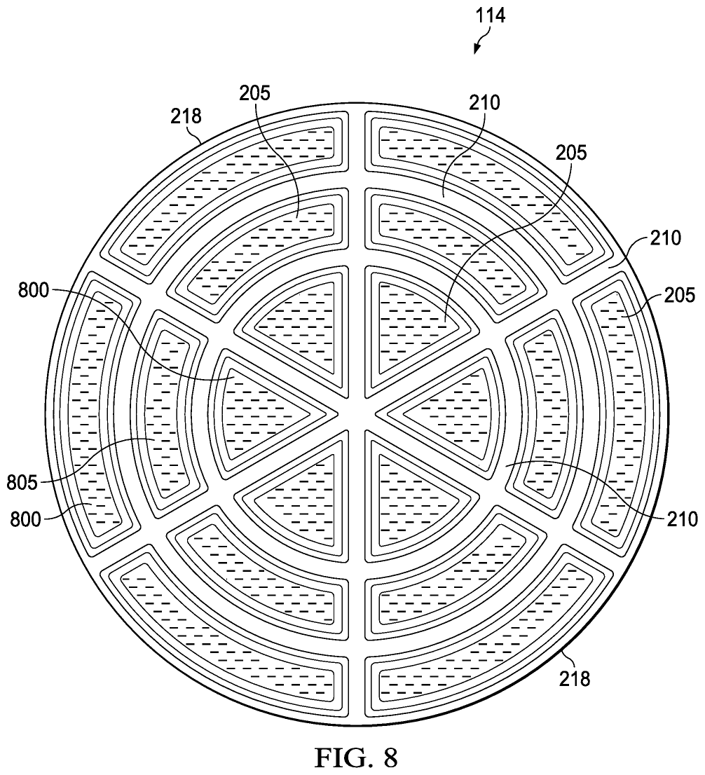

FIG. 7 is a top view of another example of the tissue interface 114, illustrating additional details that may be associated with some embodiments. In the example of FIG. 7, the tissue interface 114 is generally elliptical. Further, the interface sections 205 may have nested inner and outer edges, which may be concentric or non-concentric. In the example of FIG. 7, the interface sections 205 are bounded by seams 210 that are elliptical and concentric. In other examples, the seams 210 may define ellipses having collinear centers. The seams 210 may be spaced equally or variably from the center of the tissue interface 114. For example, the seams 210 may form outer rings that are thicker than inner rings in some embodiments.

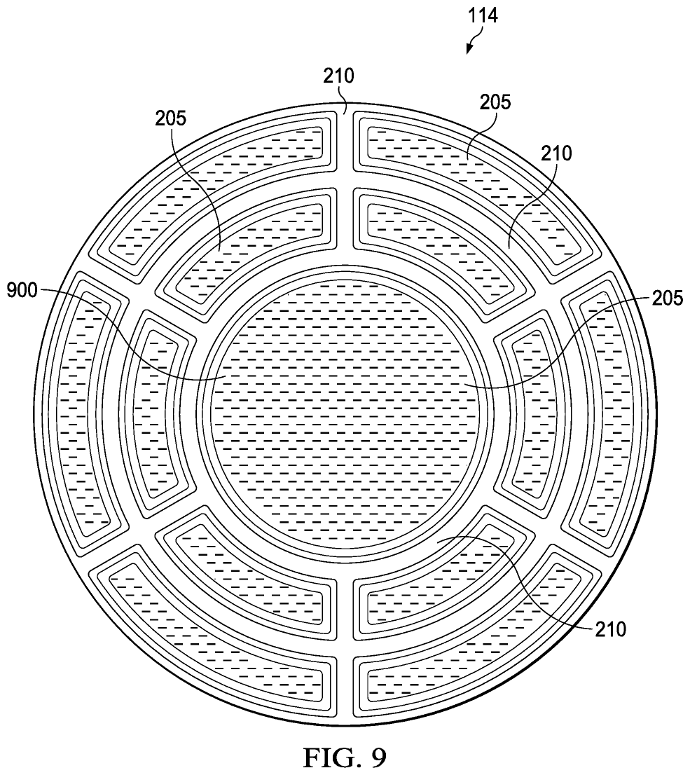

FIG. 8 is a top view of another example of the tissue interface 114, illustrating additional details that may be associated with some embodiments. In the example of FIG. 8, tissue interface 114 has a generally circular profile. Each of the interface sections 205 has at least one edge 218 that is rounded, which may be formed by seams 210 that are nested rings, for example. In the example of FIG. 8, each of the interface sections 205 has an edge 218 that is formed by seams 210 that are circular and concentric. In other examples, the seams 210 may be non-concentric. Additionally or alternatively, at least one of the seams 210 may form a chord across the tissue interface 114, which may intersect in some embodiments. As in the example of FIG. 8, two or more of the chords may intersect with a center of the tissue interface 114. Further, the seams 210 can intersect at equal angles in some examples. At least some of the seams 210 may define a sector of the tissue interface 114. Thus, at least some of the interface sections 205 may be generally triangular in shape, but with at least one rounded outer edge 218.

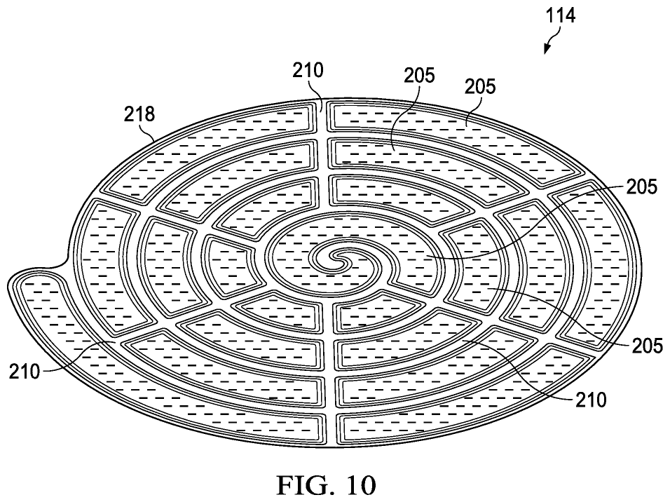

FIG. 9 is a top view of another example of the tissue interface 114, illustrating additional details that may be associated with some embodiments. In the example of FIG. 9, the tissue interface 114 has a generally circular profile. At least one of the edges 218 of each of the interface sections 205 is rounded, which may be formed by seams 210 that are nested rings, for example. In the example of FIG. 9, each of the interface sections 205 has an edge 218 that is formed by seams 210 that are circular and concentric. In other examples, the seams 210 may be non-concentric. Additional seams 210 that are linear may be formed across some of the seams 210 that are rings. In the example of FIG. 9, the seams 210 that linear do not cross the most interior of the seams 210 that are rings. Thus, the tissue interface 114 may include a generally circular central interface section and a plurality of smaller interface sections each having at least one rounded edge. Further, the interface sections 205 in the example of FIG. 9 have only acute or obtuse angles, but no right angles.

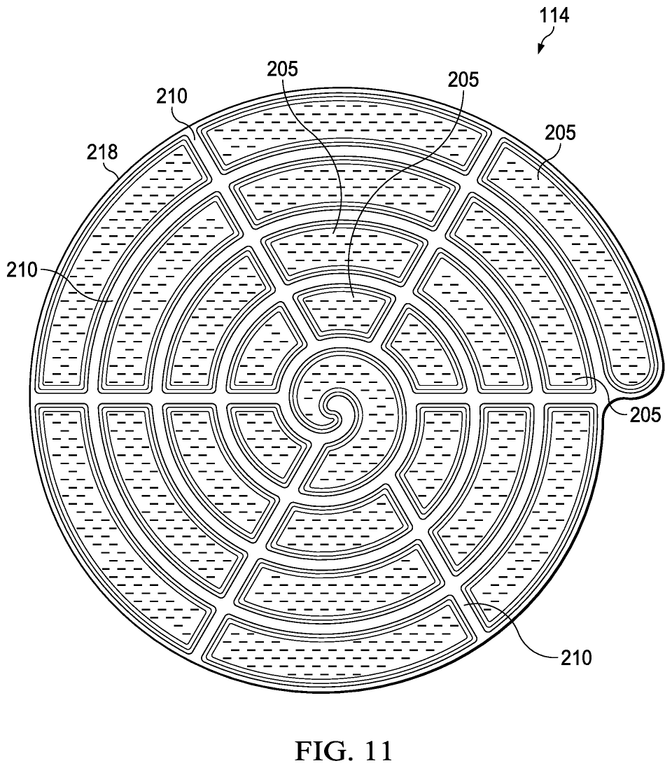

FIG. 10 is a top view of another example of the tissue interface 114, illustrating additional details that may be associated with some embodiments. In the example of FIG. 10, tissue interface 114 has a generally elliptical profile. At least one of the seams 210 in the example of FIG. 10 spirals from a center of the tissue interface 114 to an outer edge thereof. Additionally, in some embodiments, one or more seams 210 may extend radially in substantially straight lines from the center to the outer edge of the tissue interface 114. Thus, the tissue interface 114 of FIG. 10 includes a plurality of interface sections 205 each having at least one rounded edge. Further, the interface sections 205 of FIG. 10 have only acute or obtuse angles, but no right angles.

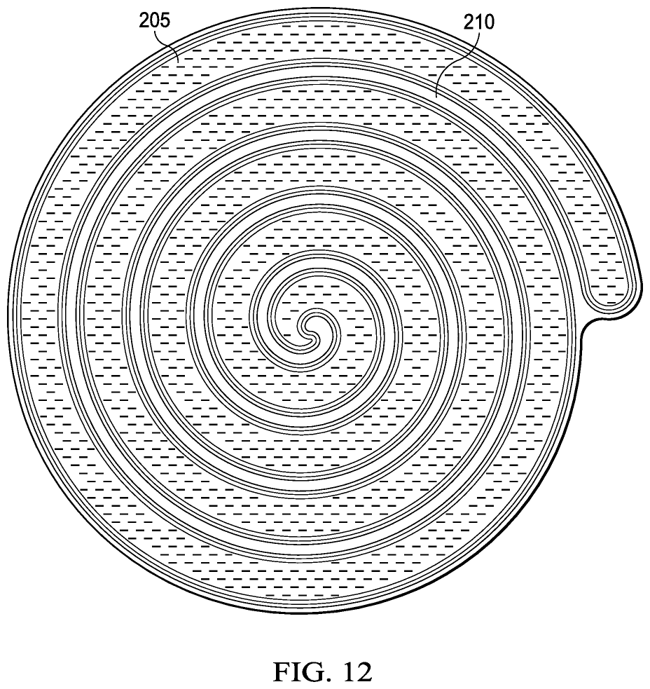

FIG. 11 is a top view of another example of the tissue interface 114, illustrating additional details that may be associated with some embodiments. In the example of FIG. 11, tissue interface 114 has a generally circular profile. At least one seam 210 of FIG. 11 spirals from a center of the tissue interface 114 to an outer edge thereof. Additionally, in some embodiments, one or more seams 210 may extend radially in substantially straight lines from the center of the tissue interface 114 to the outer edge of the tissue interface 114. Thus, the tissue interface 114 includes a plurality of interface sections 205 each having at least one rounded edge. Further, the interface sections 205 of FIG. 10 have only acute or obtuse angles, but no right angles.

FIG. 12 is a top view of another example of the tissue interface 114, illustrating additional details that may be associated with some embodiments. In the example of FIG. 12, the tissue interface 114 has a generally circular profile. The tissue interface 114 of FIG. 12 includes a long spiral shaped seam 210 extending in a spiral path from a center of the tissue interface 114 to an outer edge thereof. The seam 210 is a single long seam. The tissue interface 114 of FIG. 12 includes a single interface section 205 having only rounded edges. The tissue interface 114 may include one interface section 205, as illustrated in the example of FIG. 12 that can be an elongate shape once separated at the seam 210.

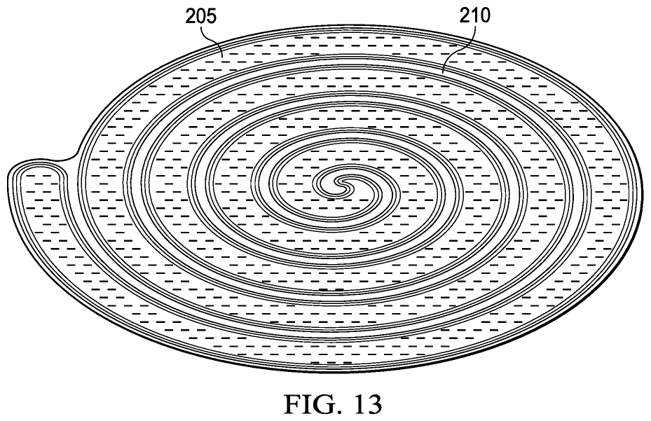

FIG. 13 is a top view of another example of the tissue interface 114, illustrating additional details that may be associated with some embodiments. In the example of FIG. 13, the tissue interface 114 has a generally elliptical profile. The tissue interface 114 includes a long spiral shaped seam 210 extending from a center of the tissue interface 114 to an outer edge thereof. The tissue interface 114 of FIG. 12 consists of a single interface section 205 having only rounded edges. The tissue interface 114 of FIG. 13 consists of one interface section 205, which can be an elongate shape once separated at the seam 210.

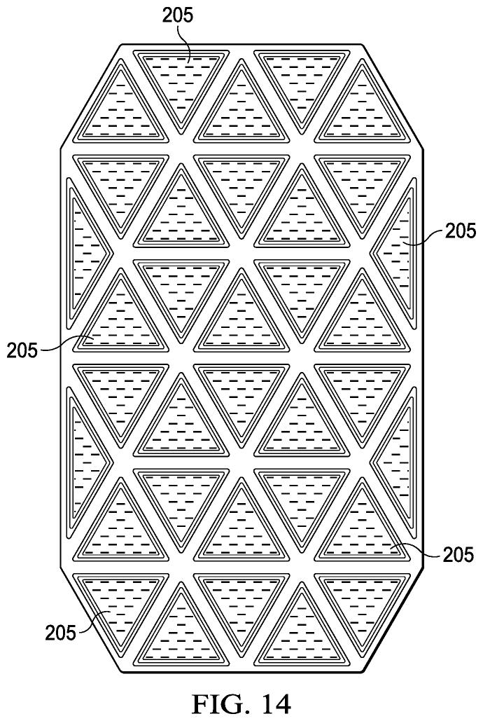

FIG. 14 is a top view of another example of the tissue interface 114, illustrating additional details that may be associated with some embodiments. As illustrated in the example of FIG. 14, the seams 210 may form polygons having only acute angles. The interface sections 205 of FIG. 14, for example, have generally triangular profiles. Vertices of the triangular profiles may be rounded in some embodiments (not shown). The triangular profiles may be equilateral triangles, isosceles triangles, or scalene triangles, for example. Moreover, as illustrated in the example of FIG. 14, the interface sections 205 may be tiled to form a tessellation.

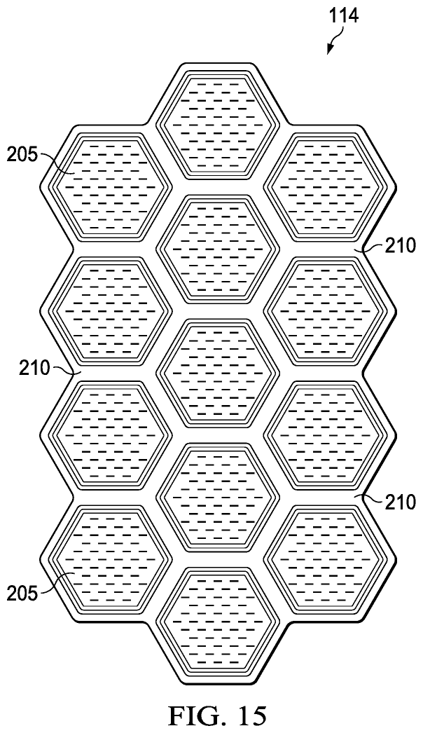

FIG. 15 is a top view of another example of the tissue interface 114, illustrating additional details that may be associated with some embodiments. As illustrated in the example of FIG. 15, the seams 210 may form other polygon profiles having only obtuse angles, such as hexagonal profiles. Some profiles, such as the hexagonal profiles of FIG. 15, may define a tessellation of interface sections 205.

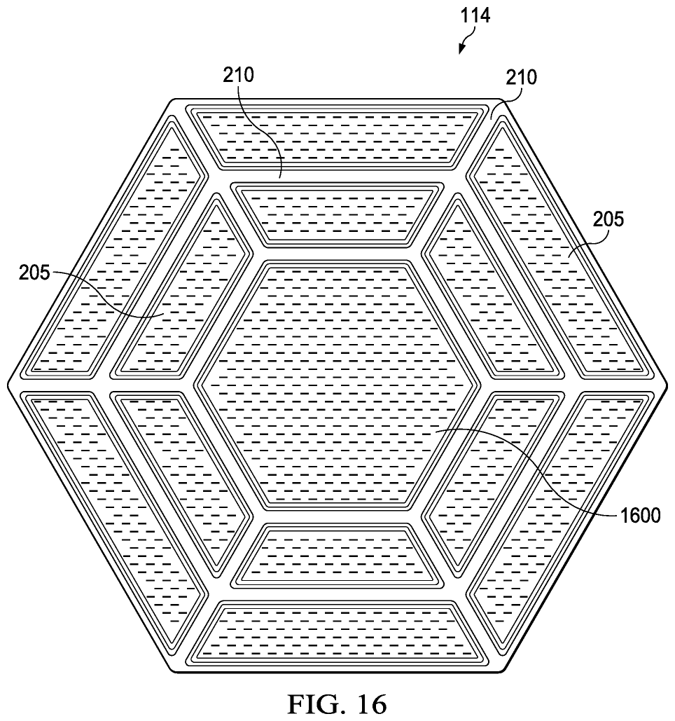

FIG. 16 is a top view of another example of the tissue interface 114, illustrating additional details that may be associated with some embodiments. In some embodiments, as shown in FIG. 16, the tissue interface 114 may have a generally hexagonal profile. For example, the seams 210 may define interface sections 205 that are nested, each of the interface sections having a hexagonal profile. Additionally, in some embodiments, seams 210 may be formed across the hexagonally shaped rings of the tissue interface 114, such that the seams 210 extend from vertices of an innermost ring to vertices of an outermost ring. In some embodiments, the seams 210 can form interface sections 205 having generally trapezoidal profiles having only acute and obtuse angles therein, and no right angles.

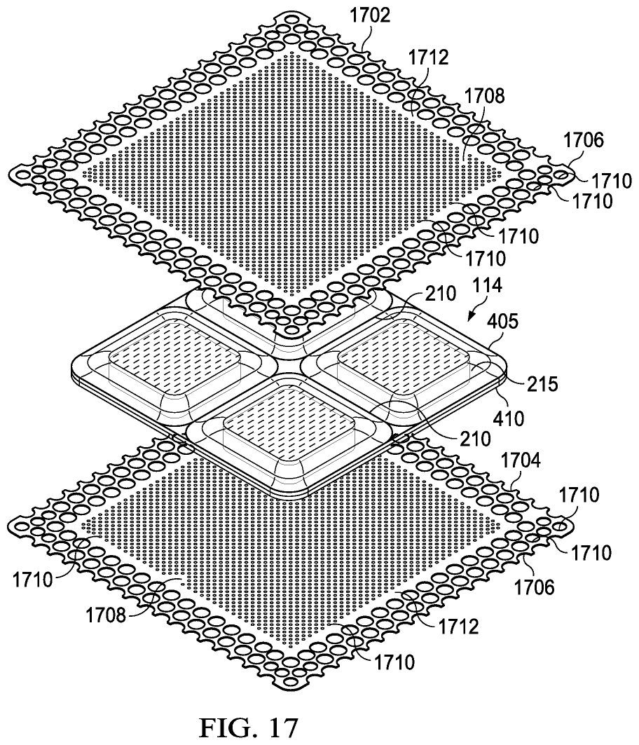

FIG. 17 is an assembly view of another example of the tissue interface 114 of FIG. 1. In the example of FIG. 17, the tissue interface 114 includes the first layer 405, the second layer 410, the manifold sections 215, a third layer 1702, and a fourth layer 1704.

The third layer 1702, the fourth layer 1704, or both may comprise or consist essentially of a soft, pliable material suitable for providing a fluid seal with a tissue site, and may have a substantially flat surface. The third layer 1702, the fourth layer 1704, or both may be a sealing layer. In some embodiments, the third layer 1702, the fourth layer 1704, or both may also be adhesive. For example, the third layer 1702 may comprise, without limitation, a silicone gel, a soft silicone, hydrocolloid, hydrogel, polyurethane gel, polyolefin gel, hydrogenated styrenic copolymer gel, a foamed gel, a soft closed cell foam such as polyurethanes and polyolefins coated with an adhesive, polyurethane, polyolefin, or hydrogenated styrenic copolymers. A silicone gel having a coating weight of about 100 g·s·m. to about 150 g·s·m. may be suitable for some applications. In some embodiments, the third layer 802, the fourth layer 804, or both may have a thickness between about 200 microns (μm) and about 1000 microns (μm). In some embodiments, the third layer 1702, the fourth layer 1704, or both may have a hardness between about 5 Shore OO and about 80 Shore OO. Further, the third layer 1702, the fourth layer 1704, or both may be comprised of hydrophobic or hydrophilic materials.

In some embodiments, the third layer 1702, the fourth layer 1704, or both may be a hydrophobic-coated material. For example, either or both may be formed by coating a spaced material, such as, for example, woven, nonwoven, molded, or extruded mesh with a hydrophobic material. The hydrophobic material for the coating may be a soft silicone, for example.

The third layer 1702, the fourth layer 1704, or both may have a periphery 1706 surrounding or around an interior portion 1708, and apertures 1710 disposed through the periphery 1706 and/or the interior portion 1708. The interior portion 1708 may correspond to a surface area of the first layer 405 or the second layer 410 in some examples. An interior border 1712 may be disposed around the interior portion 1708, between the interior portion 1708 and the periphery 1706. The interior border 1712 may be substantially free of the apertures 1710, as illustrated in the example of FIG. 17. In some examples, as illustrated in FIG. 17, the interior portion 1708 may be symmetrical and centrally disposed.

The apertures 1710 may be formed by cutting or by application of local RF or ultrasonic energy, for example, or by other suitable techniques for forming an opening. The apertures 1710 may have a uniform distribution pattern, or may be randomly distributed on the third layer 1702, the fourth layer 1704, or both. The apertures 1710 may have many shapes, including circles, squares, stars, ovals, polygons, slits, complex curves, rectilinear shapes, triangles, for example, or may have some combination of such shapes.

Each of the apertures 1710 may have uniform or similar geometric properties. For example, in some embodiments, each of the apertures 1710 may be circular apertures, having substantially the same diameter. In some embodiments, the diameter of each of the apertures 1710 may range from about 1 millimeter to about 50 millimeters. In other embodiments, the diameter of each of the apertures 1710 may range from about 1 millimeter to about 20 millimeters.

In other embodiments, geometric properties of the apertures 1710 may vary. For example, the diameter of the apertures 1710 may vary depending on the position of the apertures 1710. In some embodiments, the diameter of the apertures 1710 in the periphery 1706 may be larger than the diameter of the apertures 1710 in the interior portion 1708. For example, in some embodiments, the apertures 1710 disposed in the periphery 1706 may have a diameter ranging from about 9.8 millimeters to about 10.2 millimeters. In some embodiments, the apertures 1710 disposed in the corners may have a diameter ranging from about 7.75 millimeters to about 8.75 millimeters. In some embodiments, the apertures 1710 disposed in the interior portion 1708 may have a diameter ranging from about 1.8 millimeters to about 2.2 millimeters.

At least one of the apertures 1710 in the periphery 1706 may be positioned at an edge of the periphery 1706, and may have an interior cut open or exposed at the edge that is in fluid communication in a lateral direction with the edge. As shown in the example of FIG. 17, the apertures 1710 in the periphery 1706 may be positioned proximate to or at the edges and in fluid communication in a lateral direction with the edges. The apertures 1710 positioned proximate to or at the edges may be spaced substantially equidistant around the periphery 1706 as shown in the example of FIG. 17. Alternatively, the spacing of the apertures 1710 proximate to or at the edges may be irregular.

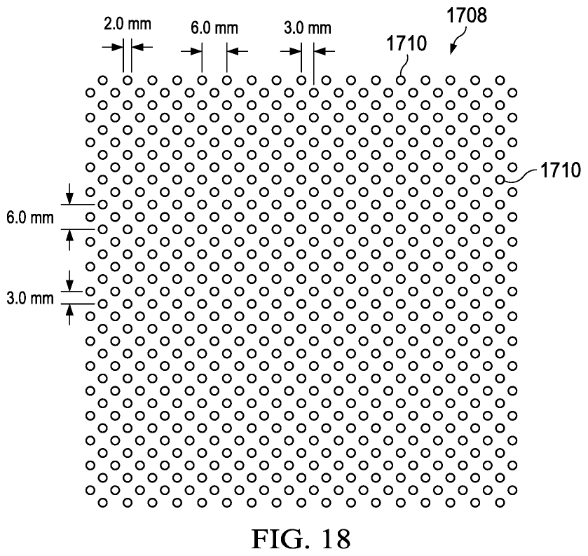

FIG. 18 is a schematic view of an example configuration of the apertures 1710, illustrating additional details that may be associated with some embodiments of the third layer 1702, the fourth layer 1704, or both.

In some embodiments, the apertures 1710 may be arranged in rows, columns, or a grid of rows and columns. The apertures 1710 may be offset in some embodiments. For example, as illustrated in FIG. 18, the apertures 1710 in one row may be offset from the apertures 1710 in adjacent rows, and the apertures in one column may be offset from the apertures in adjacent columns. In other embodiments, the apertures 1710 in adjacent rows or columns may be aligned. A pattern of the apertures 1710 may be substantially uniform in some configurations. Within each row and column, for example, the apertures 1710 may be equidistant from each other. FIG. 18 illustrates one example configuration that may be particularly suitable for many applications, in which the apertures 1710 are spaced about 6 millimeters apart along each row and column, with a 3 millimeter offset. The apertures 1710 in the interior portion 1708 of FIG. 18 have a diameter of about 2.0 mm. In some embodiments, the pattern of apertures 1710 is non-uniform.

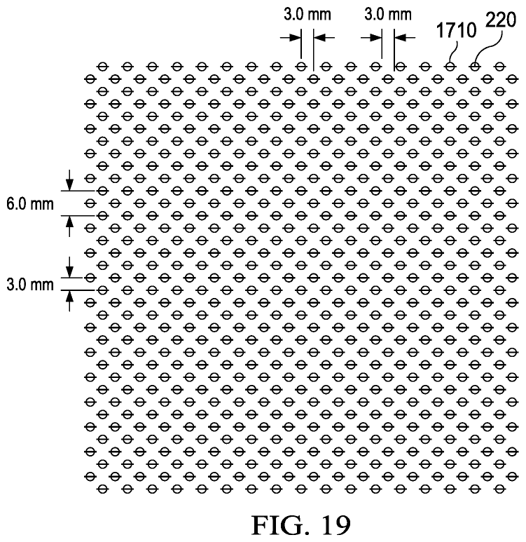

FIG. 19 is a schematic view of the example configuration of the apertures 1710 of FIG. 18 overlaid on an example configuration of the fluid restrictions 220, illustrating additional details that may be associated with some example embodiments of the tissue interface 114. For example, as illustrated in FIG. 19, the fluid restrictions 220 may be aligned, overlapping, in registration with, or otherwise fluidly coupled to at least some of the apertures 1710 in some embodiments. In some embodiments, one or more of the fluid restrictions 220 may be registered with the apertures 1710 only in the interior portion 1708, or only partially registered with the apertures 1710. The fluid restrictions 220 in the example of FIG. 19 are generally configured so that each of the fluid restrictions 220 is registered with only one of the apertures 1710. In other examples, one or more of the fluid restrictions 220 may be registered with more than one of the apertures 1710. For example, any one or more of the fluid restrictions 220 may be a perforation or a fenestration that extends across two or more of the apertures 1710. Additionally or alternatively, one or more of the fluid restrictions 220 may not be registered with any of the apertures 1710.

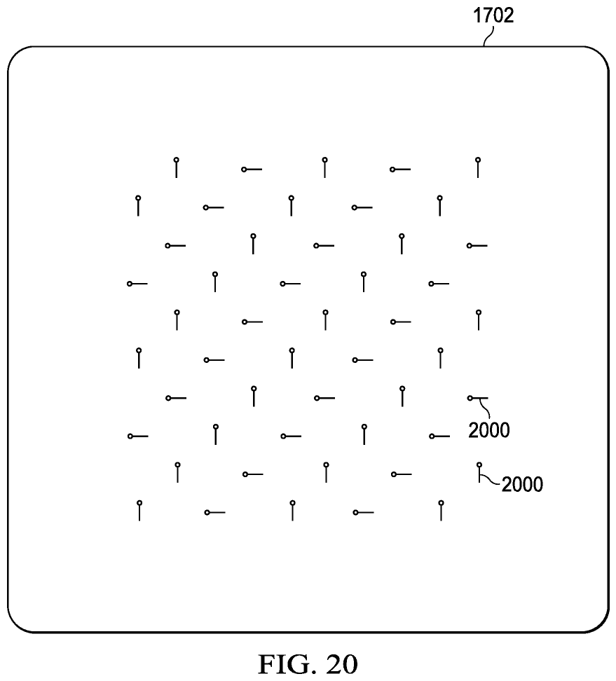

FIG. 20 is a schematic view of another example of the third layer 1702, illustrating additional details that may be associated with some embodiments. As shown in the example of FIG. 20, the third layer 1702 may have one or more fluid restrictions, such as valves 2000, instead of or in addition to the apertures 1710 in the interior portion 1708. The valves 2000 may be elastomeric.

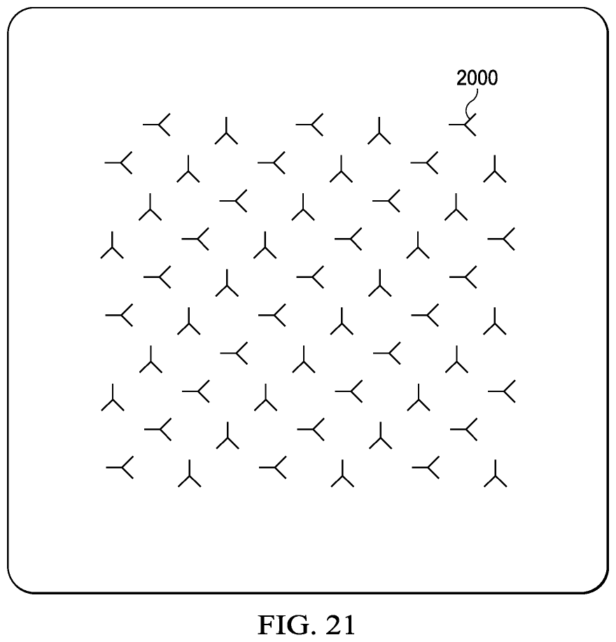

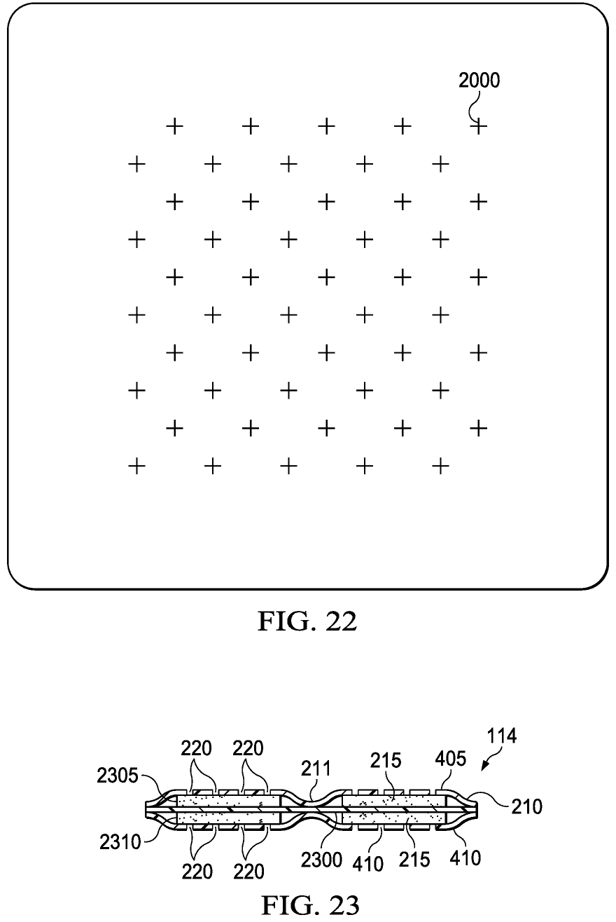

FIG. 21 and FIG. 22 illustrate other example configurations of the valves 2000, in which the valves 2000 each generally comprise a combination of intersecting slits or cross-slits. In some embodiments, shown in FIG. 21, the valves 2000 generally have a “Y” shape. In some embodiments, shown in FIG. 22, the valves 2000 generally have a cross or plus shape.

FIG. 23 is a schematic section view of another example of the tissue interface 114 that may be associated with some embodiments of the therapy system of FIG. 1. In some embodiments, as shown in FIG. 23, the tissue interface 114 may include a first manifold layer 2305 and a second manifold layer 2310. The first manifold layer 2305 may be separated from the second manifold layer 2310 by an intermediate layer 2300. In some embodiments, the intermediate layer 2300 may comprise or consist essentially of a polymer film. As illustrated in the example of FIG. 23, the first manifold layer 2305 may be disposed adjacent to the first manifold layer 2305, opposite the intermediate layer 2300. The second layer 410 may be disposed adjacent to the second manifold layer 2310, opposite the intermediate layer 2300. The first layer 405 and the second layer 410 can enclose the first manifold layer 2305, the second manifold layer 2310, and the intermediate layer 300 in some embodiments. The seams 210 can be formed between the first layer 405, the second layer 410, and the intermediate layer 2300 to enclose or form the manifold sections 215 in each of the first manifold layer 2305 and the second manifold layer 2310. Additional layers of manifold sections 215 and intermediate layers similar to intermediate layer 2300 may also be included in some embodiments. The fluid restrictions 220 can extend through each of the first layer 405 and the second layer 410.

In some embodiments, the separable forms may include a low tack adhesive applied to an outer surface thereof to aid in holding multiple separable forms in place within a wound.

In some embodiments, the tissue interface 114 may include an antimicrobial coating that is applied to the films of the tissue interface 114 during extrusion. The films may be printed or colored to aid in identification. Further, the films may be textured to include the ability to write on the separable sections. The films may be embossed with numbers for each section so that the separable sections may be counted during application and removal.

In some embodiments, a method for treating a tissue site may include excising separable sections of a dressing based upon at least one of a size and shape of the tissue site being treated. The separable sections may have at least one rounded edge. In some embodiments, the separable sections have only obtuse or acute angles, and no right angles. In some embodiments, the separable sections have only obtuse angles. In other embodiments, the separable sections have only acute angles. The method may also include applying the dressing to fill and/or cover the tissue site, and sealing the dressing to epidermis adjacent to the tissue site. The method may further include fluidly coupling the dressing to a negative-pressure source, and applying negative pressure from the negative-pressure source to the dressing.

In some embodiments, excising separable sections may comprise cutting a seam or a seal between the separable sections. In some configurations, the separable sections may be excised without exposing a manifold section inside the dressing. Each of the separable sections may have at least one of a rounded edge, an acute angle, or an obtuse angle.

The systems, apparatuses, and methods described herein may provide significant advantages. For example, in some embodiments, the seams 210 may be wide enough to allow the interface sections 205 to be cut apart or otherwise separated so as to obtain a tissue interface 114 having a desired size and shape. For example, tissue interface 114 can be sized and shaped to fill deep and/or irregular wounds by separating the interface sections 205. Moreover, some embodiments of the dressing 104 may be worn for about 3 to about 10 days (e.g., about 7 days). For example, elongate spiral shaped interface sections 205 may more efficiently fill long and/or deep wounds. For example, interface sections 205 having more rounded shapes may, when torn along the seams, not leave acute corners that may irritate a wound. In some embodiments, the interface sections 205 may be formed in bright colors to make it easier to see the interface sections 205 in the wound and reduce the odds of the interface sections 205 being left in the wound.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 102, the container 112, or both may be eliminated or separated from other components for manufacture or sale. In other example configurations, the controller 108 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A dressing for treating a tissue site with negative pressure, the dressing comprising:
a manifold comprising a first surface and a second surface opposite the first surface;
a first layer adjacent to the first surface and a second layer adjacent to the second surface, the first layer and the second layer each comprising a polymer film;
a plurality of fluid restrictions in the polymer film adjacent to at least the first surface; and
a plurality of bonds between the first layer and the second layer, the plurality of bonds defining separable sections of the manifold, each of the separable sections of the manifold having at least one rounded edge.

2. The dressing of claim 1, wherein the plurality of bonds form seams between the separable sections of the manifold.

3. The dressing of claim 1, wherein the plurality of bonds form seams having a width of at least 2 millimeters between the separable sections of the manifold.

4. The dressing of claim 1, wherein the plurality of bonds form seams having a width of at least 2 millimeters and less than 5 millimeters between the separable sections of the manifold.

5. The dressing of claim 1, wherein the manifold comprises perforations aligned with the bonds.

6. The dressing of claim 1, wherein the manifold comprises perforations between the separable sections.

7. The dressing of claim 1, wherein the manifold comprises:
perforations aligned with the bonds; and
sacrificial joints between the separable sections.

8. The dressing of claim 1, wherein the manifold comprises perforations having a width of about 10 millimeters.

9. The dressing of claim 1, wherein the plurality of bonds form a seal between the separable sections of the manifold.

10.-40. (canceled)

41. The dressing of claim 1, wherein:
the plurality of fluid restrictions are distributed across the polymer film in rows and columns that are mutually parallel;
the rows are spaced about 3 millimeters on center; and
the fluid restrictions in each of the rows are spaced about 3 millimeters on center.

42. (canceled)

43. (canceled)

44. The dressing of claim 1, wherein the plurality of fluid restrictions comprise or consist essentially of elastomeric valves in the polymer film that are normally closed.

45. The dressing of claim 44, wherein the elastomeric valves are fenestrations.

46. The dressing of claim 1, wherein the first layer is coextensive with the second layer.

47. The dressing of claim 1, wherein the plurality of fluid restrictions in the polymer film adjacent to the first surface and the second surface.

48. The dressing of claim 1, wherein the plurality of bonds have a tear strength in a range of about 10 newtons to about 25 newtons.

49. A dressing for treating a tissue site with negative pressure, the dressing comprising:
a manifold comprising a first surface and a second surface opposite the first surface;
a first layer adjacent to the first surface and a second layer adjacent to the second surface, the first layer and the second layer each comprising a polymer film;
a plurality of fluid restrictions in the polymer film adjacent to at least the first surface; and
a means for bonding the first layer to the second layer to form separable sections of the manifold, the separable sections having at least one rounded edge.

50.-52. (canceled)

53. The dressing of claim 49, wherein the means for bonding the first layer to the second layer comprises welds.

54. The dressing of claim 49, wherein the means for bonding the first layer to the second layer comprises one or more sacrificial bonds.

55.-65. (canceled)

66. An apparatus for providing negative-pressure treatment to a tissue site, the apparatus comprising:
a manifold comprising a first surface and a second surface opposite the first surface;
a first layer adjacent to the first surface and a second layer adjacent to the second surface, the first layer and the second layer each comprising a polymer film;
a plurality of fluid restrictions in the polymer film adjacent to at least the first surface;
a plurality of bonds between the first layer and the second layer, the plurality of bonds defining separable sections of the manifold, the separable sections each having at least one rounded edge; and
a negative-pressure source fluidly coupled to the manifold.

67.-84. (canceled)
